SEQUENCE LISTING (3 sequences)

SEQ ID NO: 1 - DNA, 635 bp, Artificial Sequence
  Homo sapien chomosome with flanking vector nucleotides
ggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttaattatctccacgttgaaaaagactgaagtccaataagtgcctcaaaggctgctatagttgtaaggaagggtccaagagggaaagattccagtcttccatttgtaaatcaatcagatctctgttacagggaaatgtgtgtgtacatgtgtgtgtgtgtggggggggggtgttgggagtgcatgcataggaattaaggcacttgggtcatcagcaaagcctggtgcgttttctgtaacctctaaacagctggtagaaagaatccaataagtcaattccttgagaagattagcaattagggggttgtatgagtctttttgggttcaatagctctcccatcatatattggggaaatttaattaattctctgaagcttgagtattctatagtctcacctaaatagcttggcgtaatcatggtcatagctgtttcctgtgtgaaattgttatccgcctcacaattccacacaacatacgag

SEQ ID NO: 2 - DNA, 565 bp, Artificial Sequence
  Homo sapien chomosome with flanking vector nucleotides
ggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagctttgtgagcacagatcacagggattttgttttctactgatcccctagtgcacagaacaggctgggcttgtagctggcacacaatgcatagaatggacaatctattaagtctattttgtgtgctggtcaatggaacccagcagaaatgatggatgggtttagaaattggctgcaaggaacaagggcttcttattttggagacaagatagtatgaagggacagtttcattctgacaacagcttataggtaatcatttttgaaaatgaattttgtatgtggcctcaagatacagaacttggtgcaagcttgagtattctatagtctcacctaaatagcttggcgtaatcatggtcatagctgtttcctgtgtgaaattgttatccgctcacaattccacacaacatacgag

SEQ ID NO: 3 - DNA, 580 bp, Artificial Sequence
  Homo sapien chomosome with flanking vector nucleotides
ggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgcaattacaagaagtgcaaatactttcagatttgttttggcgtgatgtaccgtagatttcttttaccaatgttgactatccactgaagtctggactctaaaattcttcaacattcaaattcactgcctgaggaaaaacacatgggtagaacagagaatgagactatggcatttaattattaaatcaatgcatgcctatggtaggaactatgcaaagaattcctcaaactacagtattgcagataggctgcttgcgcaagaacaattgcctgacatgactgtctgcaccaatgcactaacaccaactactgcaataagcttgagtattctatagtctcacctaaatagcttggcgtaatcatggtcatagctgtttcctgtgtgaaattgttatccgctcacaattccacacaacatacgag

(19) United States
(12) Patent Application Publication
(10) Pub. No.: US 2015/0329898 A1
(43) Pub. Date: Nov. 19, 2015

(54) Mate-Pair Sequences from Large Inserts

(71) Applicant: Amplicon Express, Pullman, WA (US)

(72) Inventors: Robert Bogden, Pullman, WA (US); Suresh Iyer, Pullman, WA (US); Quanzhou Tao, Pullman, WA (US)

(73) Assignee: Amplicon Express, Pullman, WA (US)

(21) Appl. No.: 14/281,821

(22) Filed: May 19, 2014

(51) Int. Cl. C12Q 1/68

(52) U.S. Cl. CPC C12Q 1/6806; C12Q 1/6869

(57) ABSTRACT

This disclosure describes modified methods and compositions of matter pertaining to mate pair sequencing. In an implementation of the invention, mate pair sequence data is generated without cloning in a host system. In an implementation of the invention, a nucleic acid fragment is ligated into a vector. The vector containing the inserted nucleic acid fragment is digested with a restriction endonuclease that cuts at two or more places on the insert but does not cut the vector. Following the restriction endonuclease digest, the vector with portions of the nucleic acid fragment remaining after the restriction endonuclease digestion is again ligated. This ligation connects the cut ends of the insert to each other. The re-ligated product may be sequenced to obtain sequence data for both a “right” and “left” side of the originally inserted nucleic acid fragment.

### BACKGROUND

Using Mate-pair libraries for Next Generation DNA is used for de novo DNA sequencing of genomes and re-sequencing known genomes. Mate-pair sequencing allows the determination of two “reads” of sequence from two places on a single polynucleotide molecule without sequencing the entire molecule. In some situations, the mate-pair approach allows for more information to be gained from sequencing two stretches of nucleic acid sequences, each “n” bases in length than from sequencing “n” bases from each of two independent nucleic acid sequences in a random fashion. For example, with the use of appropriate software tools for the assembly of sequence information it is possible to make use of the knowledge that the mate-pair sequences are known to occur on a single duplex, and are therefore linked or paired in the genome by a separation of some distance—even if an exact length of the separation is unknown. This information can aid in computer-assisted assembly of numerous shorter sequences by aligning the shorter sequences with the mate-pair sequence to correctly assemble a larger sequence. It is possible to use mate-pair sequence information to build a scaffold which connects two contiguous DNA sequences (contigs) across a gap comprised of difficult/impossible to sequence motifs (i.e., repeated regions).

One technique for generating mate pairs uses DNA strands between 35 and 45 kilobases (kb) ligated into a fosmid vector that are transfected into E. coli using a lambda phage. The fosmid vector containing the DNA strand of interest is reproduced as the E. coli replicate and the resulting fosmid DNA is then isolated from the E. coli for and used in NxSeq™ mate-pair library construction and subsequent Next Generation sequencing.

### BRIEF SUMMARY

Various embodiments disclosed herein are generally directed towards compositions and methods for making mate pairs and mate-pair libraries. A nucleic acid “mate pair” is a length of nucleic acid sequence that includes two tags (nucleic acid sequences) that are generated from a polynucleotide of interest. Generally, each tag is a separate part of the polynucleotide of interest, allowing sequencing on the separate tags while still allowing one to associate the sequences of the two tags together during later sequence analysis because it is known that the tags are ultimately derived from the same continuous polynucleotide fragment.

In an aspect of the present invention, a double-stranded polynucleotide is prepared for sequencing. A circular nucleic acid molecule that has a vector and an insert is digested with a restriction endonuclease. In an implementation the vector is a DNA vector. The vector may be a bacterial artificial chromosome (BAC), a yeast artificial chromosome (YAC), a P1-derived artificial chromosome (PAC), a transformation-competent artificial chromosome (TAC), another type of artificial chromosome, or a different type of vector. The vector may be capable of holding long inserts such as inserts longer than about 20 kb. The vector may also be capable of holding shorter inserts or existing as a circular nucleic acid molecule without an insert. In an aspect of the invention, insert may be a sequence of interest. In an aspect of the invention, the insert may be genomic DNA, mitochondrial DNA, chloroplast DNA, synthetic DNA, cloned DNA, another type of DNA, or combination thereof.

In an aspect of the invention, digestion with the restriction endonuclease may be performed to completion. The restriction endonuclease may be capable of recognizing its corresponding restriction site on methylated DNA, such as but not limited to, genomic DNA. The restriction endonuclease may be Nsil, EcoRI, or another restriction endonuclease. The vector portion of the circular nucleic acid molecule lacks restriction sites for the restriction endonuclease. Thus, digestion with the restriction endonuclease does not cut the vector portion of the circular nucleic acid molecule. The insert in the circular nucleic acid molecule has at least two restriction sites for the restriction endonuclease. Thus, the insert will be cut in two or more places by the restriction endonuclease.

In an aspect of the invention, the nucleic acid molecule is ligated following the restriction endonuclease digestion. Since the insert has at least two sites that are cut by the restriction endonuclease, a middle portion of the insert is removed. Ligation connects one end of the insert that remains attached to the vector to the other end of the insert that is also connected to the vector thereby creating a smaller circular nucleic acid molecule that includes the vector and both of the ends of the insert. The ligation reaction may follow the restriction endonuclease digestion reaction without a buffer change. Thus, in an aspect of the invention, the digestion and the ligation may occur in a same buffer solution. Implementations of the invention may remove or inactivate the restriction enzyme following the digestion and prior to the ligating. Moreover the digestion and the ligation may occur without introducing the nucleic acid molecule, at any stage, into a host cell. Thus, the insert retains the same chemical composition because the insert is not introduced into a host cell to be re-manufactured.

In an aspect of the invention, a mate pair may be prepared by digesting a cloning vector construct with a restriction endonuclease then ligating the digested, cloning vector construct. The restriction endonuclease recognizes corresponding restriction sites on methylated genomic DNA. The restriction endonuclease may be Nsil, EcorRI, or another restriction endonuclease. The cloning vector construct contains an artificial chromosome and a genomic DNA insert. The artificial chromosome lacks any restriction sites for the restriction endonuclease. The genomic DNA insert has at least two restriction sites for the restriction endonuclease. In an implementation of the invention, the artificial chromosome is a BAC, a YAC, a PAC, a TAC, or another kind of artificial chromosome. In an implementation of the invention, the artificial chromosome may be pECBAC1, pBELO11, pCC1BAC, pindigoBAC-5, and pBACe36.

The ligating joins one end of the genomic DNA cut by the restriction endonuclease to the other end of the genomic DNA cut by the restriction endonuclease thereby re-ligating the cloning vector construction without a middle portion (i.e., removing a portion of the insert between the restriction endonuclease sites on the genomic DNA).

In an aspect of the invention, the digesting and the ligating occur in a same buffer solution. In some implementations, the restriction endonuclease is inactivated or removed following the digestion and prior to the ligating. In an aspect of the invention, the digestion and the ligating may occur without introducing the cloning vector construct into a cell.

In an aspect, the invention comprises a circular nucleic acid molecule. The circular nucleic acid molecule may include a vector capable of stably holding DNA inserts of at least 20 kb. The circular nucleic acid molecule may lack an origin of replication and may lack a restriction site for a restriction endonuclease. The circular nucleic acid molecule also includes two ends of a DNA fragment but omits a middle portion of the DNA fragment between the two ends of the DNA fragment. Both of the ends of the DNA fragment are ligated to each other at a restriction site of a restriction endonuclease, thereby forming a smaller vector construct due to omission of the middle sequence.

### BRIEF DESCRIPTION OF THE FIGURES

FIG. 1 shows a flowchart of proceeding from high-molecular weight nucleic acid to analyses sequence data according to an embodiment of the invention.

FIG. 2 shows a graphical illustration of steps included in obtaining nucleic-acid fragments of a desired molecular weight range.

FIG. 3 shows a schematic of an insert prior to insertion into a vector.

FIG. 4 shows a schematic of restriction endonuclease sites present on a circular polynucleotide molecule generated by the ligation of the insert and vector shown in FIG. 3.

FIG. 5 shows a schematic of the circular polynucleotide molecule of FIG. 4 following digestion with a restriction endonuclease.

FIG. 6 shows a schematic of a circularized polynucleotide molecule generated by ligating the digestion product of FIG. 5.

FIG. 7 shows an image of a Pulsed Field gel of circularized vectors containing inserts.

FIG. 8 shows an image of a gel and standard electrophoresis of circularized vectors containing inserts that have been subjected to restriction endonuclease digest according to an embodiment of the invention.

FIGS. 9A, 9B, and 9C show DNA sequences generated from an embodiment of the invention.

### DETAILED DESCRIPTION

### Definitions

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2^{nd} Ed. 2006).

Unless defined otherwise, all technical and scientific terms used herein have the same meaning as commonly understood by those of ordinary skill in the art to which the invention belongs. For the purposes of the present invention, the following terms are defined below.

As used herein, the terms “polynucleotide” and “nucleic acid” designates mRNA, RNA, synthetic RNA, cRNA, rRNA, cDNA. mtDNA, cpDNA, genomic DNA, synthetic DNA, and combinations thereof. These terms typically refer to polymeric forms of nucleotides of at least 10 bases in length, either ribonucleotides, deoxynucleotides, or a chimeric mixture thereof. The terms include both single and double stranded forms of DNA and RNA. The terms include both methylated DNA and non-methylated DNA. Included within the terms “polynucleotide” and “nucleic acid” are segments and smaller fragments of such segments, and also recombinant vectors, including, for example, artificial chromosomes, plasmids, cosmids, phagemids, phage, viruses, and the like. Unless denoted otherwise, whenever a polynucleotide sequence is represented, it will be understood that the nucleotides are in 5′ to 3′ order from left to right and that “A” denotes (deoxy)adenosine, “C” denotes (deoxy)cytidine, “G” denotes (deoxy)guanosine, “T” denotes (deoxy)thymidine and “U” denotes uridine.

Polynucleotides may be single-stranded (coding or antisense) or double-stranded, and may be DNA or RNA molecules. Additional coding or non-coding sequences may, but need not, be present within a polynucleotide of the present invention, and a polynucleotide may, but need not, be linked to other molecules and/or support materials.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has effects on the efficiency and strength of hybridization between nucleic acid strands.

As used herein “tag,” “sequence tag” or “tag sequence” refer to a subsequence of a polynucleotide of interest.

A “mate pair,” also known as “MP,” “paired-end,” tag mate pair,” or “paired tag,” contains two tags (each a nucleic acid sequence) that are from each end region of a polynucleotide of interest. Thus, a paired tag includes sequence fragment information from two parts of a polynucleotide. In some embodiments, this information can be combined with information regarding the polynucleotide's size, such that the separation between the two sequenced fragments is known to at least a first approximation. This information can be used in mapping where the sequence tags came from.

As used herein, “starting polynucleotide” denotes the original polynucleotide from that the polynucleotide of interest can be derived from. For example, a sample from a cell, where the starting polynucleotide is fragmented into acceptable sizes to serve as polynucleotides of interest. Of course, the options and variations of the starting polynucleotide are at least as broad as the options for the polynucleotide of interest.

A nucleic acid sequence, fragment, or paired tag clone having “compatible ends” means that the ends are compatible with joining to another nucleic acid sequence, fragment or paired tag clone as provided herein. Compatible ends can be “sticky ends” having a 5′ and/or 3′ overhang, or alternatively, compatible ends can be “blunt ends” having no 5′ and/or 3′ overhang. In generally, sticky ends can permit sequence-dependent ligation, whereas blunt ends can permit sequence-independent ligation. Compatible ends can be produced by any known methods that are standard in the art. For example, compatible ends of a nucleic acid sequence can be produced by restriction endonuclease digestion of the 5′ and/or 3′ end.

The terms “restriction endonuclease” or “restriction enzyme” refer to enzymes that cut DNA at or near specific recognition nucleotide sequences known as “restriction sites” or “restriction recognition sites.” Restriction endonuclease includes both enzymes that are able to recognize and cut methylated DNA and enzymes that only recognize un-methylated DNA. Methylated DNA includes dam methylation, dcm methylation and CpG methylation. Restriction endonucleases may recognize four, six, or eight nucleotide long restriction sites. These types of restriction endonucleases are referred to a 4-cutters, 6-cutters, and 8-cutters respectively.

By “enzyme reactive conditions” it is meant that any necessary conditions are available in an environment (i.e., such factors as temperature, pH, adenosine triphosphate (ATP), and lack of inhibiting substances) which will permit the enzyme to function. Enzyme reactive conditions can be either in vitro, such as in a test tube, or in vivo, such as within a cell.

A restriction endonuclease may be combined with a substrate and exposed to enzyme reactive conditions that cause the restriction endonuclease to perform a “partial digestion” or a “complete digestion.” A partial digestion is a reaction catalyzed by a restriction endonuclease under conditions that result in less than all possible restriction sites being cut. A complete digestion or “digestion to completion” is a reaction that results in all or substantially all of the possible restriction sites being cut.

“Inactivation” of a restriction endonuclease refers to altering the enzyme, substrate, or enzyme reactive conditions to cease or to substantially reduce the ability of the enzyme to cut DNA or RNA. Inactivation includes inactivation by heat or cold. For restriction endonucleases that do not recognize methylated restriction sites, inactivation may also be achieved by methylating a DNA substrate.

By “corresponds to” or “corresponding to” is meant a restriction site having a nucleotide sequence that is recognized by a restriction endonuclease. The restriction endonuclease may cut the DNA or RNA at a location within the restriction site or at another location nearby. The “cutting site” is the location at which the phosphate backbone of a polynucleotide sequence is cut by a restriction endonuclease. The cutting site is usually, but not always, the same as the restriction site.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

By “isolated” is meant material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

By “obtained from” is meant that a sample such as, for example, a polynucleotide extract or polypeptide extract is isolated from, or derived from, a particular source, such as a desired organism or a specific tissue within a desired organism. “Obtained from” can also refer to the situation in which a polynucleotide or polypeptide sequence is isolated from, or derived from, a particular organism or tissue within an organism.

“Transformation” refers to the permanent, heritable alteration in a cell resulting from the uptake and incorporation of foreign DNA into the host-cell; also, the transfer of an exogenous gene from one organism into the genome of another organism.

The word “vector,” as used herein, is a polynucleotide molecule capable of being ligated to and stably holding a separate polynucleotide molecule referred to as an “insert.” A vector may include one or more unique restriction sites. The vector may be longer or shorter than the insert. Both the vector and the insert may be the same or different type of nucleic acid (e.g., gDNA, mtDNA, cpDNA, cDNA, plasmid, cosmid, fosmid, BAC, YAC, P1, TAC, synthetic DNA). “Vector” includes both naturally-derived polynucleotides and synthetic polynucleotides.

As use herein, “vector” includes, but is not limited to, “cloning vectors” that may be, for example, derived from a plasmid, bacteriophage, yeast, or virus, into which a polynucleotide can be inserted or cloned. A cloning vector can be capable of autonomous replication in a defined host cell including a target cell or tissue or a progenitor cell or tissue thereof, or be integrable with the genome of the defined host such that the cloned sequence is reproducible. Accordingly, the cloning vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The cloning vector can contain any means for assuring self-replication. Alternatively, the cloning vector can be one which, when introduced into the host cell, is integrated into the genome and replicated together with the chromosome(s) into which it has been integrated. Such a vector may comprise specific sequences that allow recombination into a particular, desired site of the host chromosome. A cloning vector system can comprise a single vector or plasmid, two or more vectors or plasmids, which together contain the total DNA or RNA to be introduced into the genome of the host cell, or a transposon. The choice of the cloning vector will typically depend on the compatibility of the cloning vector with the host cell into which the cloning vector is to be introduced. The cloning vector is preferably one which is operably functional in a host cell. The cloning vector can include a reporter gene, such as a green fluorescent protein (GFP), which can be either fused in frame to one or more of the encoded polypeptides, or expressed separately. The cloning vector can also include a selection marker such as LacZ gene that can be disrupted by an insert causing the colony growth to be white, or lack of an insert wherein the colony turns blue when appropriate chemicals are included in the growth media (e.g., IPTG and Xgal). The cloning vector can also include a selection marker such as an antibiotic resistance gene that can be used for selection of suitable transformants.

As used herein, the term “vector construct” refers to a vector that is ligated to an insert to form a circular nucleic acid molecule.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but can alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the method used.

As used herein, the term “polymerase chain reaction” (“PCR”) refers to the method of K. B. Mullis that is known to persons having ordinary skill in the art and described in U.S. Pat. Nos. 4,683,195 and 4,683,202. PCR includes a method for increasing the number of copies a segment of a DNA sequence of interest sequence without cloning or purification. This process for amplifying the polynucleotide of interest consists of introducing a large excess of two oligonucleotide primers to the DNA mixture containing the desired polynucleotide of interest sequence, and unincorporated nucleotides (dNTP's), followed by a precise sequence of thermal cycling in the presence of a DNA polymerase. The two primers are complementary to their respective strands of the double stranded polynucleotide of interest sequence. To effect amplification, the mixture is denatured and the primers then annealed to their complementary sequences within the polynucleotide of interest molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired polynucleotide of interest. The length of the amplified segment of the desired polynucleotide of interest is determined by the relative positions of the primers with respect to each other, and therefore, this length is a controllable parameter. By virtue of the repeating the process, the method is referred to as the “polymerase chain reaction” (hereinafter “PCR”). Because the desired amplified segments of the polynucleotide of interest sequence become the predominant sequences (in terms of concentration) in the mixture, they are said to be “PCR amplified.”

### Mate-Pair Sequencing

The present disclosure, therefore, relates generally to methods and articles of manufacture pertaining to generation of mate-pair sequence data.

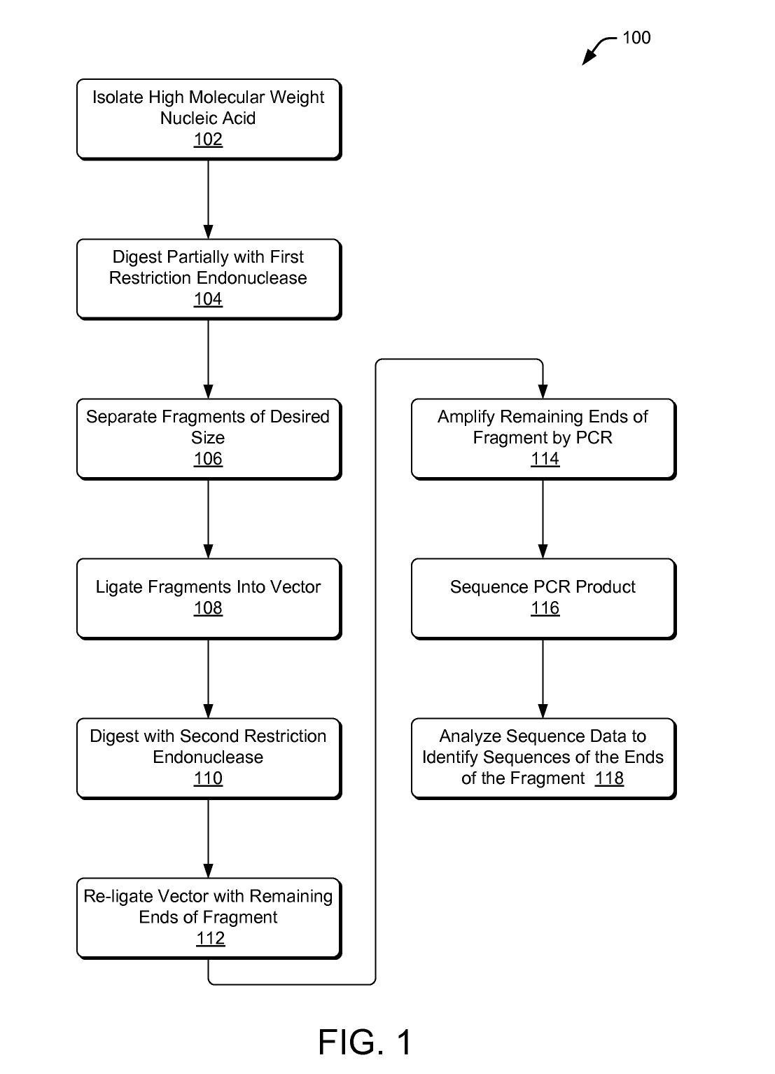

FIG. 1 shows a method 100 of preparing a vector, processing the vector, and sequencing a portion of the vector. At 102, high molecular weight nucleic acid is isolated by any appropriate technique. In one implementation, the high molecular weight nucleic acid may be isolated from cell or tissue sample of an organism. One technique for preparing high molecular weight nucleic acid that will be known to the person having ordinary skill in the art is described in Zhang, et al., Preparation of megabase-size DNA from plant nuclei. The Plant Journal. 7 (1), 175-184. (1995). The high molecular weight nucleic acid may be DNA, RNA, or a combination thereof. In an implementation, the high molecular weight nucleic acid is genomic DNA.

At 104, the high molecular weight nucleic acid is digested with a first restriction endonuclease. The digestion may be a partial digestion achieved by stopping the digestion at various time points to obtain different levels of completion. The digestion creates multiple fragments of the high molecular weight nucleic acids with a variety of sizes. The digestion may be performed while the high molecular weight nucleic acid remains embedded in agarose immediately following extraction of the high molecular weight nucleic acid. Persons having ordinary skill in the art will understand how to vary reaction conditions based on the type of high molecular weight nucleic acid, the specific restriction endonuclease, and desired frequency of digestion. Some restriction endonucleases that may be used are [list of a 3-5]. One technique for preparing high molecular weight nucleic acid and subjecting the high molecular weight nucleic acid to a restriction endonuclease digest is described in Tao, et al., Construction of a full bacterial artificial chromosome (BAC) library of Oryza sativa genome. Cell Research. 4,127-133 (1994).

At 106, fragments of the high molecular weight nucleic acid are separated by size. One technique for separating fragments of high molecular weight nucleic acids is pulsed-field gel electrophoresis (PFGE). Nucleic acid fragments of the desired weight ranges may be selected by excision from the gel used to run the PFGE. The nucleic acid fragments of the desired weights may be isolated from the gel using known techniques such as dialysis, electroluting, and enzymatic digestion of the gel (e.g., agarase enzyme that degrades agar). Specific examples of techniques for separating nucleic acid fragments by size using PFEG are described in Tao, et al. Cloning and stable maintenance of DNA fragments over 300 kb in Escherichia coli with conventional plasmid-based vectors. Nucleic Acids Research. Vol. 26, No. 21,4901-4909 (1998) and in Tao, et al., Construction of a full bacterial artificial chromosome (BAC) library of Oryza sativa genome. Cell Research. 4,127-133 (1994).

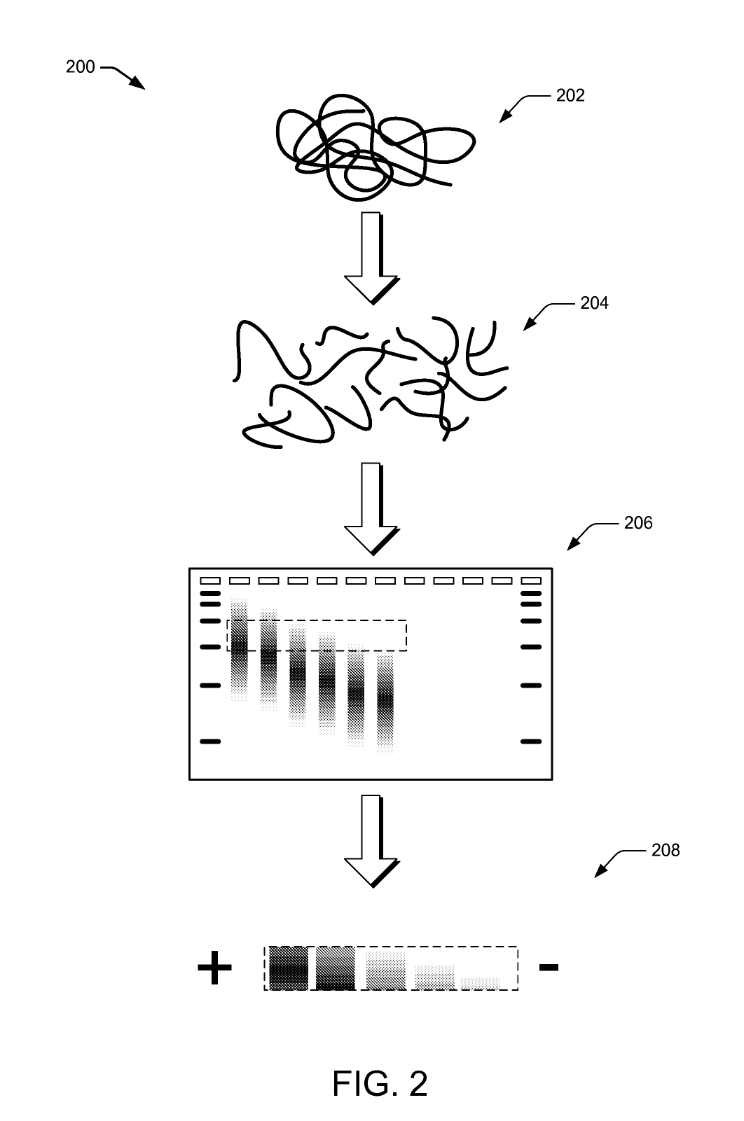

FIG. 2 illustrates an embodiment 200 of implementing 102-106 of FIG. 1. Following digestion with a restriction endonuclease the high molecular weight nucleic acid 202 is cut into multiple shorter fragments 204. The fragments may be separated on a PFGE gel 206. Markers on the PFGE gel 206 are used to identify the approximate molecular weight of the fragments spread over a lane of the PFGE gel 206. Fragments of a desired size range may be cut from the gel. In an implementation the size range may be about 50 kb, about 75 kb, about 100 kb, about 125 kb, about 150 kb, about 175 kb, about 200 kb, about 300 kb, about 400 kb, about 500 kb, about 600 kb, about 700 kb, about 800 kb, about 900 kb, or about 1 mb. Application of an electric field 208 may be used to remove the fragments from the gel 206 by electrolution.

Returning to FIG. 1, at 108 fragments of the high molecular weight nucleic acid generated by the procedures above are ligated into a vector. The chemical composition of the insert DNA in the vector is from the original organism of interest, the insert DNA has never been introduced into a host cell or copied by a host cell. Illustrative vectors that may be used include, but are not limited to, pECBAC1, pBELO11, pCC1BAC, pindigoBAC-5, and pBACe36. Since the fragments were cut by the first restriction endonuclease, the ends of the fragments may be ligated with other nucleic acids that were also cut with the same restriction endonuclease. Thus, the vector may be digested with the same restriction endonuclease or created so that the ends of the vector correspond to ends cut with the same restriction endonuclease. The restriction endonuclease may create sticky ends or blunt ends when cutting a nucleotide sequence. A person of ordinary skill in the art will be able to readily identify an appropriate ligase and reaction conditions based on the vector, the fragments of the high molecular weight nucleic acid, and the restriction endonuclease used to make the cuts. Illustrative procedures that may be used for this ligation are described in Tao, et al., Construction of a full bacterial artificial chromosome (BAC) library of Oryza sativa genome. Cell Research. 4, 127-133 (1994), Zhang, et al., Preparation of megabase-size DNA from plant nuclei. The Plant Journal. 7 (1), 175-184. (1995), and Tao, et al. Cloning and stable maintenance of DNA fragments over 300 kb in Escherichia coli with conventional plasmid-based vectors. Nucleic Acids Research. Vol. 26, No. 21, 4901-4909 (1998).

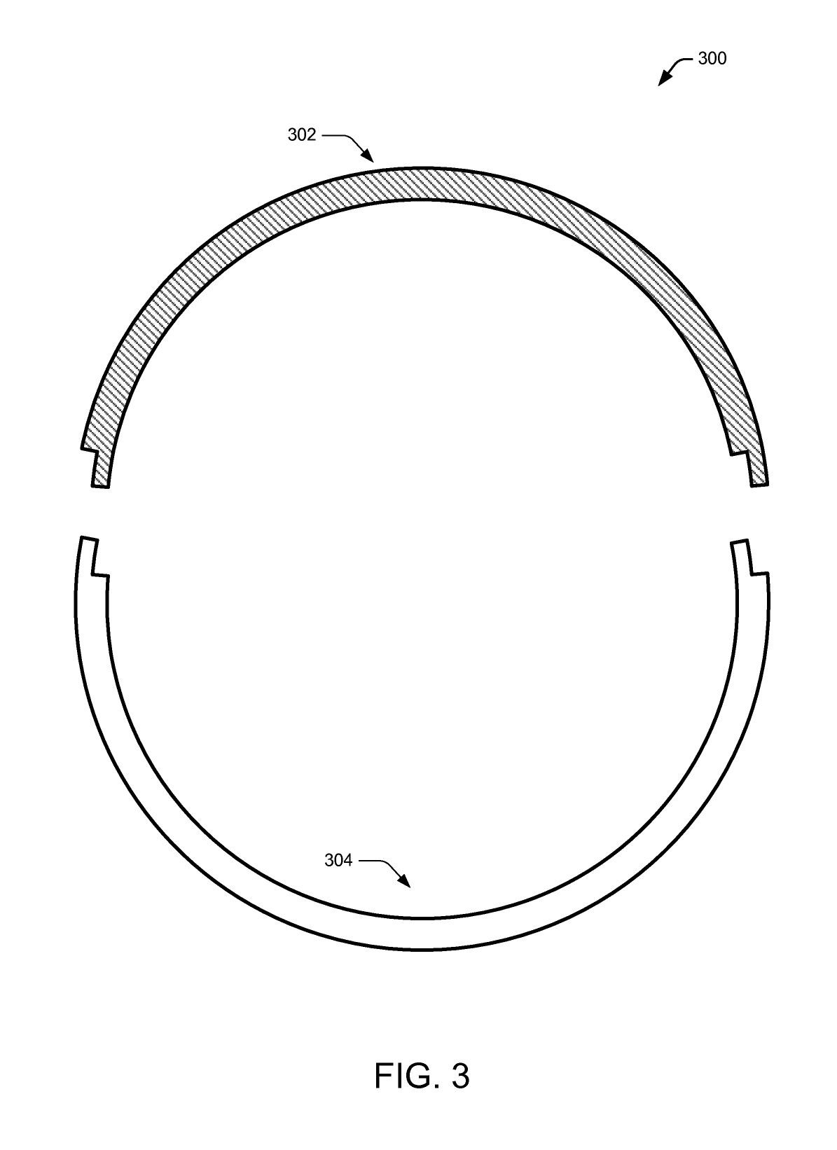

FIG. 3 shows a schematic 300 of ligation between a nucleic acid sequence of interest 302 and vector 304. Although the insert 302 and the vector 304 are shown with sticky ends the invention may also by using restriction endonucleases that create blunt ends. The end product of the ligation is a library of circular, double-stranded polynucleotide molecules containing the various insert 302 created by the restriction endonuclease digest at 104.

At 110, in FIG. 1, the vector containing the insert is digested with a second restriction endonuclease different from the first. The sequence of the vector will be known and the second restriction endonuclease is selected to not cut the vector. The sequence of the insert may be unknown when, for example, the insert is derived from genomic DNA. However, for large inserts (e.g., 10-1,000 kb) there is a reasonable likelihood that multiple restriction sites will be present. For example, assuming a random sequence of nucleotides a 4-cutter would likely cut every 256 bp, a 6-cutter would likely cut every 4 kb, and an 8-cutter would likely cut every 66 kb. Restriction endonucleases that may be used include [list of a few 6-cutters that act on methylated DNA].

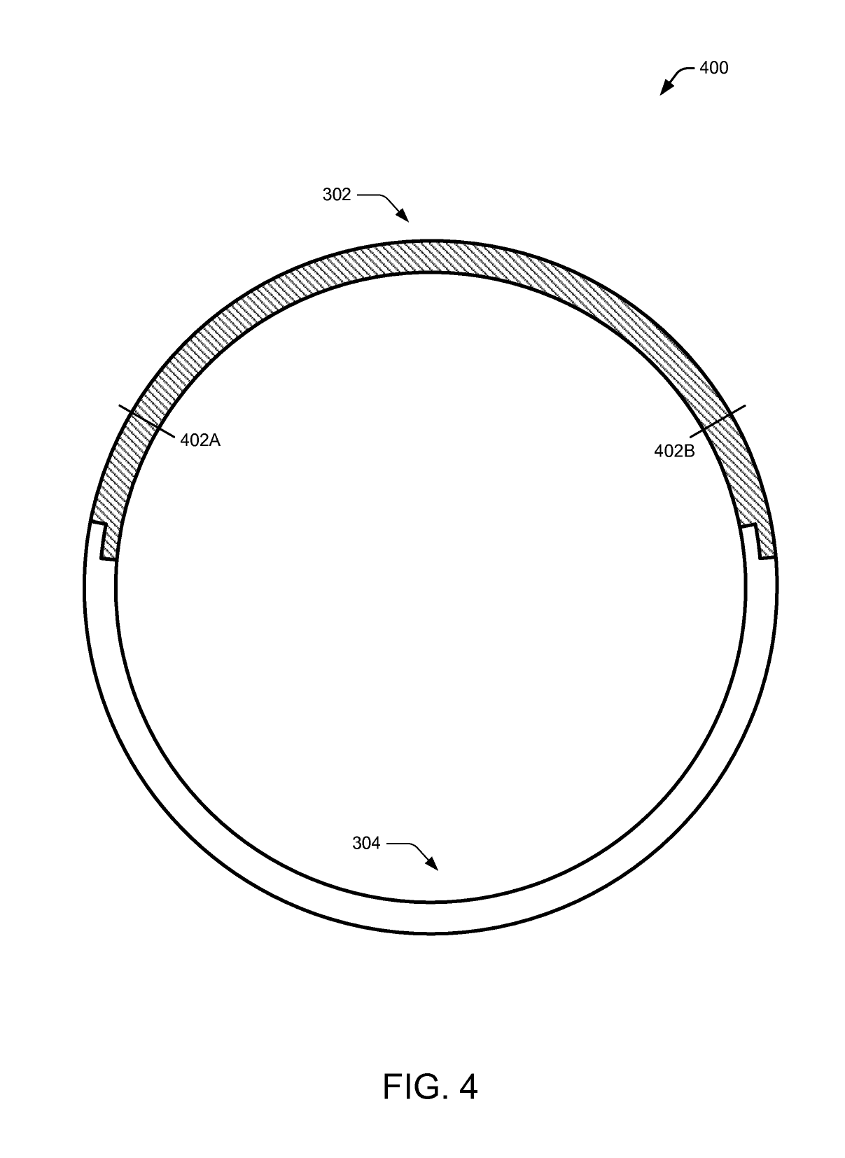

FIG. 4 shows a schematic 400 of the insert 302 and the vector 304 following ligation. The insert 302 has at least two restriction sites 402A and 402B for the second restriction endonuclease. The insert 302 may contain additional restriction sites 402 for the same restriction endonuclease. Note that the vector 304 portion of the circular polynucleotide molecule does not contain any restriction sites for that restriction endonuclease.

In an embodiment, this restriction endonuclease digestion of 110 from FIG. 1 may be performed as a complete digest. During the digestion, a middle portion of the insert is cut out from the remainder of the circular polynucleotide molecule. As will be appreciated by a person having ordinary skill in the art, the restriction endonuclease digest may be performed at standard reaction conditions for the selected restriction endonuclease. The digestion reaction may be stopped after complete digestion prior to re-ligation.

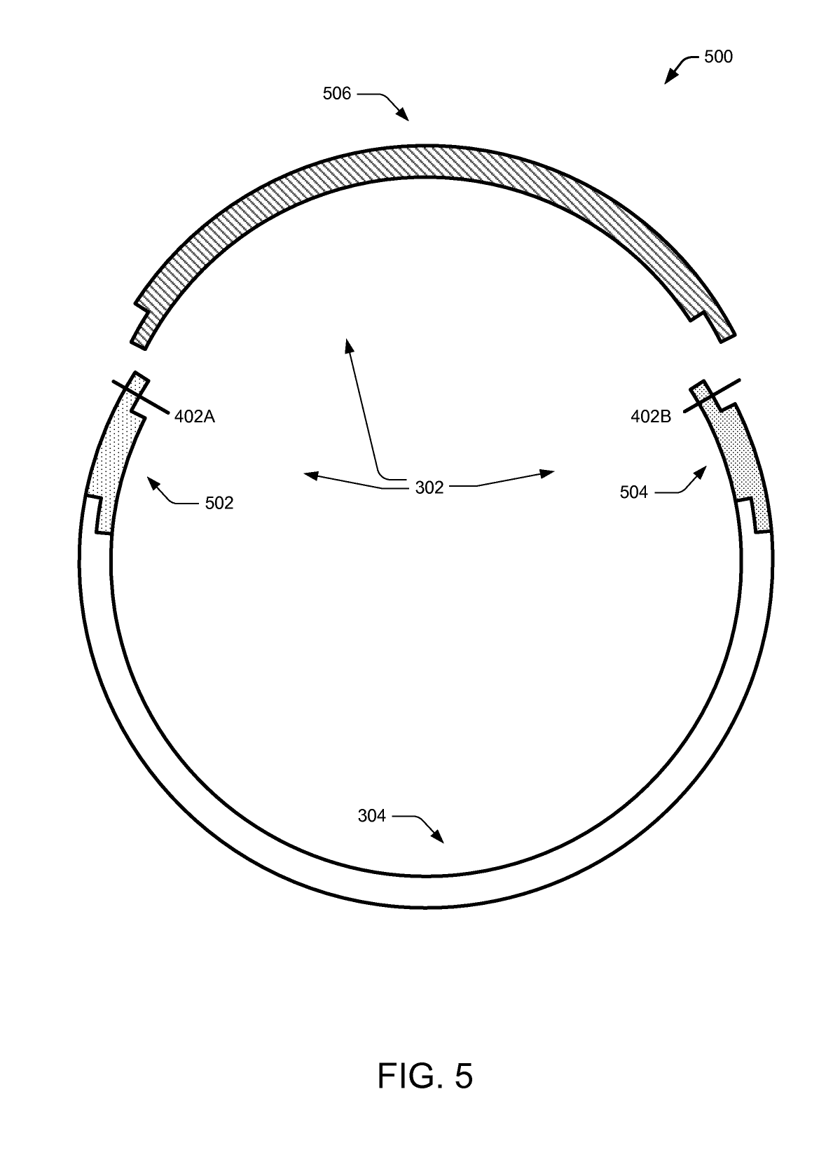

FIG. 5 shows a schematic 500 of the circular polynucleotide molecule shown in FIG. 4 following digestion at restriction sites 402A and 402B. The insert 302 is cut into a first end 502 and second end 504 that both remain attached to the vector 304. A middle portion 506 of the vector 304 is separated from the remainder of the circular polynucleotide molecule. Presence of additional restriction sites 402A and 402B within the middle portion 506 of the insert 302 would result in the middle portion 506 being further cut into smaller pieces. However, the size of the first end 502 and the second and 504 of the insert are not affected by the presence of additional restriction sites 402 in the middle portion 506 of the insert 302. Following the restriction endonuclease digest, the polynucleotide molecule is linearized into a double-stranded polynucleotide comprising the first end 502 of the insert 302, the vector 304, and the second end 504 of the insert 302.

Returning to FIG. 1, at 112, the vector 304 with the attached ends 502 and 504 of the insert is re-ligated. The re-ligation may be performed by any conventional technique. Re-ligation will also recircularize the vector 304 and the attached ends 502 and 504. In an implementation, the ends created by digestion at restriction sites 402A and 402B are compatible cohesive ends that can be ligated into a circular nucleic acid molecule. Examples of ligation techniques that may be used are described in Tao, et al., Construction of a full bacterial artificial chromosome (BAC) library of Oryza sativa genome. Cell Research. 4,127-133 (1994), Zhang, et al., Preparation of megabase-size DNA from plant nuclei. The Plant Journal. 7 (1), 175-184. (1995), and Tao, et al. Cloning and stable maintenance of DNA fragments over 300 kb in Escherichia coli with conventional plasmid-based vectors. Nucleic Acids Research. Vol. 26, No. 21,4901-4909 (1998).

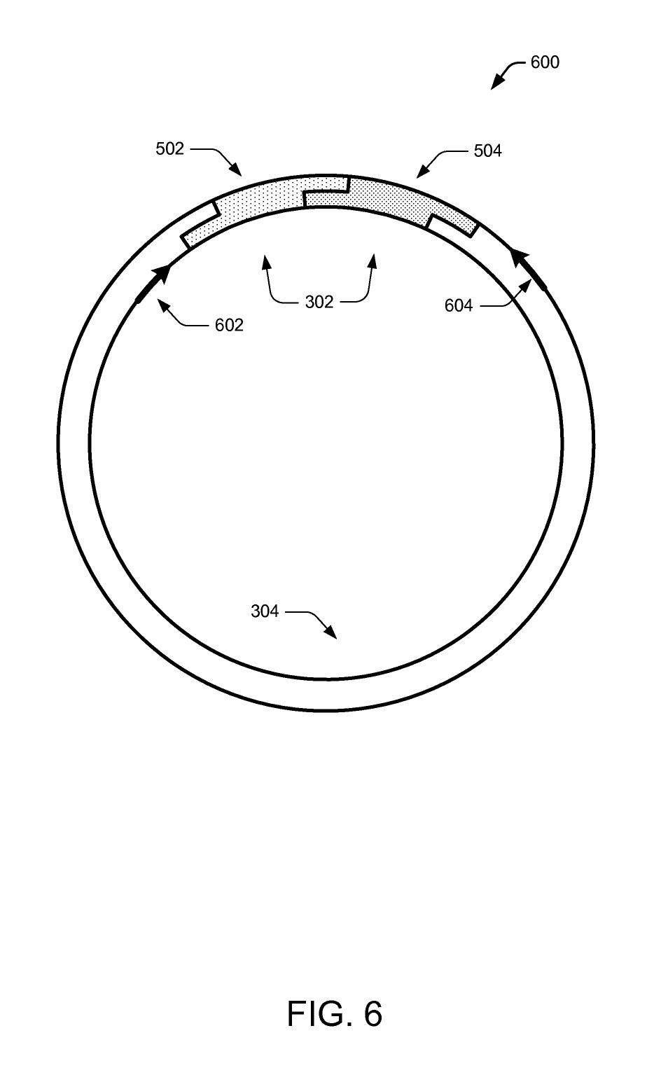

FIG. 6 shows a schematic 600 of a circular polynucleotide molecule following re-ligation. The vector 304 remains joined to the first end 502 and the second end 504 of the insert 302. The two ends 502 and 504 are now joined to each other forming a circular, double-stranded polynucleotide molecule that omits the middle portion 506 of the insert 302. The vector 304 may also include at least one primer site 602 and 604 on either side of the ends of the insert 502 and 504 suitable for polymerize chain reaction (PCR) primer binding. The priming sites 602 and 604 are present in the original vector 306 but omitted from other drawings for the sake of clarity. Thus, re-ligation provides a template for performing PCR on the ends 502 and 504. Note that since the vector 304 is not transfected/transformed into a host cell presence or absence of an origin of replication (or other functional element found on a conventional plasmid vector) on the vector 304 does not affect the other aspects of this invention.

At 114 in FIG. 1, the remaining ends of the fragment are amplified by PCR using primer sites present in the vector. PCR may be performed by any conventional technique. In an implementation, the steps performed to prepare the vector and inserted fragment for sequencing do not involve transforming or transfecting cells with the vector. Thus the original, high molecular weight nucleic acid fragments may retain epigenetic modifications. Additionally with this technique, potential bias introduced by cloning a cloning vector and insert into a host cell is not present.

At 116, the PCR product is sequenced. Suitable sequencing techniques include next-generation sequencing (NGS), and any techniques developed in the future for determining sequence of a polynucleotide molecule. For example, NGS hardware and systems produced by Illumina, Inc. of San Diego, Calif. or by Pacific Biosciences of Melo Park, Calif. may be used for sequencing. Although PCR amplification prior to sequence is described in an implementation of this invention, it is envisioned that PCR may be omitted when used with other sequencing technologies that read single nucleic acid molecules.

At 118, the sequence data generated 116 is analyzed to identify nucleotide sequences of the ends of the fragment. Techniques for analyzing sequence data generated from mate-pair sequence are known to persons skilled in the art. Sequence assembly software can build scaffolds with estimate gaps of “the middle” between two mate pair sequences. One suitable sequence assembly software product is ALLPATHS-LG discussed in Gnerre, et al., High-quality draft assemblies of mammalian genomes from massively parallel sequence data. Proceedings of the National Academy of Sciences USA. (2010). Sequence data generated using the techniques described above may be filtered to find the second restriction site and parsed into “left end” and “right end” of the DNA fragment.

### Conclusion

The terms “a,” “an,” “the” and similar referents used in the context of describing the invention (especially in the context of the following claims) are to be construed to cover both the singular and the plural, unless otherwise indicated herein or clearly contradicted by context.

As will be understood by one of ordinary skill in the art, each embodiment disclosed herein can comprise, consist essentially of, or consist of its particular stated element, step, ingredient, or component. Thus, the terms “having,” “has,” “contain,” “containing,” “include,” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds) and provided literal support for and includes the end points of 10 and 100.

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. These broad, intermediate, and narrow numerical ranges should be applied not only to the specific values, but should also be applied to differences between these specific values.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to publications, patents and/or patent applications (collectively “references”) throughout this specification. Each of the cited references is individually incorporated herein by reference for their particular cited teachings.

In closing, it is to be understood that the embodiments of the invention disclosed herein are illustrative of the principles of the present invention. Other modifications that may be employed are within the scope of the invention. No attempt is made to show structural details of the invention in more detail than is necessary for the fundamental understanding of the invention, the description taken with the drawings and/or examples making apparent to those skilled in the art how the several forms of the invention may be embodied in practice. Thus, by way of example, but not of limitation, alternative configurations of the present invention may be utilized in accordance with the teachings herein. Accordingly, the present invention is not limited to that precisely as shown and described.

### EXAMPLES

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Example 1

This example illustrates creation of DNA vectors with large genomic DNA inserts. gDNA was isolated from a human sample by embedding 5×10⁷/mL cells in low melting-point agarose plugs and the gDNA was purified in the agarose. Subsequent cell lysis and DNA purification were performed in situ on the agarose plugs by diffusing proteinase K which degrades proteins and leaves behind intact, high molecular weight gDNA. Specifically, the agarose plugs containing the high molecular weight gDNA were placed in a lysis buffer (0.5 M Na₂EDTA. 2H₂O, 10 mM Tris base, 1% (w/v) N-lauroylsarcosine sodium salt ,and 0.5 mg/mL proteniase K) for 24-48 h at 50° C. with gentle shaking. Following lysis, the agarose plugs containing HMW DNA were dialysised once in 0.5 M EDTA, pH 9.0-9.3 for 1 h at 50° C., once in 0.05 M EDTA, pH 8.0 for 1 h on ice, and then stored in 0.05 M EDTA, pH 8.0 at 4° C. Before digestion of the DNA, the agarose plugs containing HMW DNA were dialysised three times in 10-20 volumes of ice-cold TE (10 mM Tris-HCl, pH 8.0, 1 mM EDTA, pH 8.0) plus 0.1 mM phenylmethyl sulfonyl fluoride (PMSF) and three times in 10-20 volumes of ice cold TE on ice, 1 h/each dialysis. The digestion mixture was: water 31 μl, agarose plugs containing HMW DNA of about 50 μl, 10× enzyme buffer 10 μl, and 40 mM spermidine 5 μl.

After incubation on ice for 1 h, 4 μl of Hindil of each enzyme dilution series was added for partial digestion of human gDNA. After an additional 1 hour incubation on ice to allow the enzyme to access the DNA in the agarose, the reaction mixture was increased to the recommended temperature for HindIII activity (37° C.). After incubating the agarose plugs at 37° C. for 10 min. The reaction was stopped by adding 1/10 volume of 0.5 M EDTA, pH 8.0. The partially digested gDNA fragments were size-selected on 1% pulsed-field low melting point agarose gels in 0.5×TBE (45 mM Trizma base, 45 mM boric acide, and 1 mM EDTA, pH 8.3). The PFGE conditions were BLK1, 6V/cm, linear, initial time 90 s, final time 90 s, 11° C. for 18 hours and BLK2, 4V/cm, linear, initial time 5 s, final time 15 s, 11° C., for 6 hours. A range of sizes were selected between 100 Kb and 200 Kb.

A conventional plasmid vector, pECBAC1, was used for cloning the gDNA. pECBAC1 vector is about 7.5 kb in size, has the replicon of ColE1 and exists at 1 chromosomal equivalents in E. coli. The plasmid vector was prepared by digestion with 100 units of HindIII for one hour at 37° C. The plasmid vector was treated with 1 unit of Calf Intestinal Phosphatase (CIP) for every 1 pmol of vector DNA ends (about 2.5 μg for a 7.5 kb plasmid) and incubated at 37° C. for 60 minutes. Subsequently vector DNA was purified with gel extraction.

One to three hundred nanograms of the partially digested gDNA was ligated to the HindIII digested pECBAC1 (molar ratio of 5 to 1, pECBAC1 in excess) with T7 DNA ligase at 16° C. overnight. This created a library of vectors containing random fragments of high molecular-weight human gDNA.

The ligated vectors were transformed into E. coli cells by electroporation at 350 V, 330 μF capacitance, low ohm impedance, and fast charge rate with resistance set to 4000Ω.

Colorless clones were inoculated in 1 ml of LB broth containing suitable antibiotics and grown at 37° C. with shaking at 250 r.p.m. overnight. The cells were harvested and the DNA was isolated by alkaline lysis using the technique described above. The DNA was digested to completion with NotI (100 ng DNA digested with 5 units of NotI at 37° C. for 2 hours) to release the insert DNA. Twenty-eight samples were selected for pulsed-field gel electrophoresis on a 1% agarose gel in 0.5×TBE at an initial pulse time of 5 s, a final pulse time of 15 s, 120°, 6 V/cm and 14° C. for 16 h. The gel was stained in ethidium bromide, destained in water for 30 min and photographed.

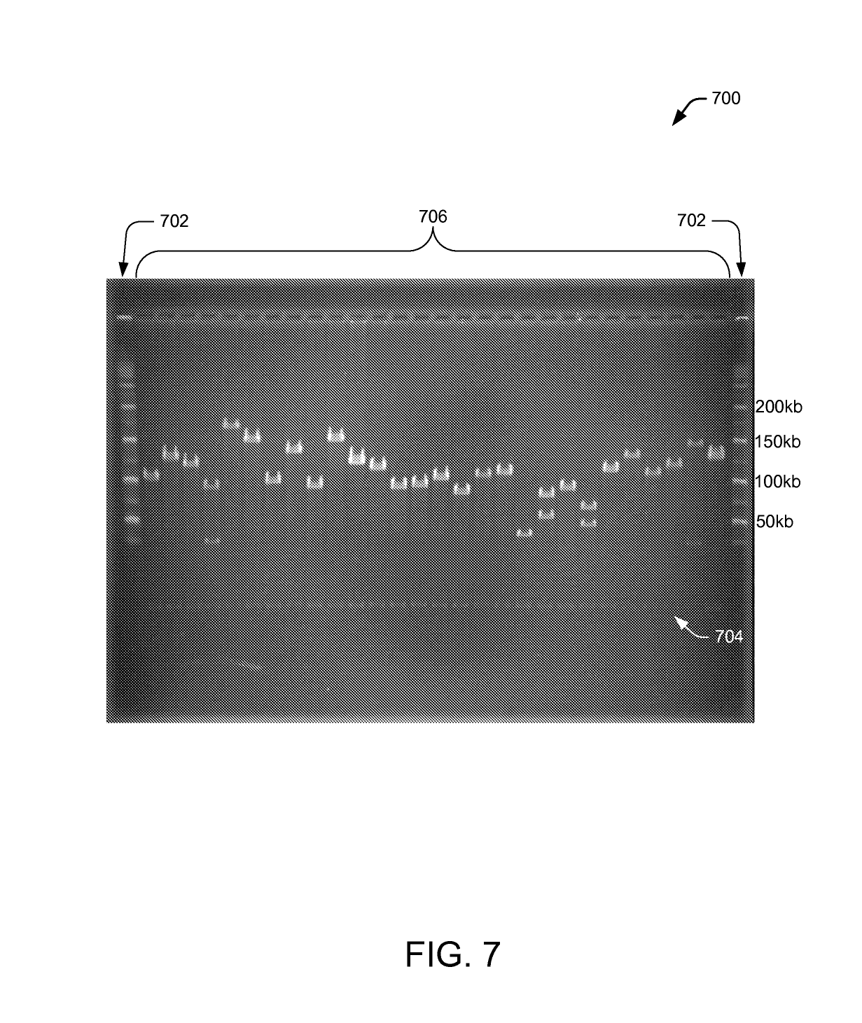

FIG. 7 shows a gel 700 generated by the above procedure. The marker lanes 702 contain a 25 kb ladder. The 7.5 kb pECBAC1 vector without an insert is shown by the faint bands 704 towards the bottom of the gel 700. The lanes 706 containing 28 samples with molecular weights ranging from about 25 kb to about 175 kb with an average size of about 100 kb. Thus, the above procedure generates high-molecular weight (i.e., average 100 kb) gDNA fragments from human genomic DNA that are capable of being inserted (and later removed as shown in gel 700) into a vector.

### Example 2

This example illustrates reduced size of the vectors generated in Example 1 following ligation of the vector DNA and human gDNA insert DNA. pECBAC1 vectors with gDNA inserts were created as described in Example 1. However, instead of transforming the vectors into E. coli as described in Example 1, the ligated vectors were digested to completion with Nsil (140 ng DNA digested with 20 units of Nsil at 37° C. for 2 hours). Following digestion the non-circular polynucleotides were re-circularized by ligation. Ligation was performed by adding 1200 units (NEB) T7 DNA ligase to 60 ul of the Nsil digestion mixture and allowing to sit at 16° C. for 5 hours.

The re-ligated vectors were then transformed into E. coli DH10b host cells by electroporation at 350 V, 330 μF capacitance, low ohm impedance, and fast charge rate with resistance set to 4000Ω. Colorless clones were inoculated in 1 ml of LB broth containing suitable antibiotics and grown at 37° C. with shaking at 250 r.p.m. overnight. The cells were harvested and the DNA was isolated by alkaline lysis using the technique described above. The DNA was again digested to completion with NotI (100 ng of DNA digested with 5 units of NotI at 37° C. for 2 hours). Forty-four samples were selected for standard gel electrophoresis on a 0.8% agarose gel in 1×TBE 100 Volts for 2 hours at room temperature. The gels was stained in ethidium bromide, destained in water for 30 min, and photographed.

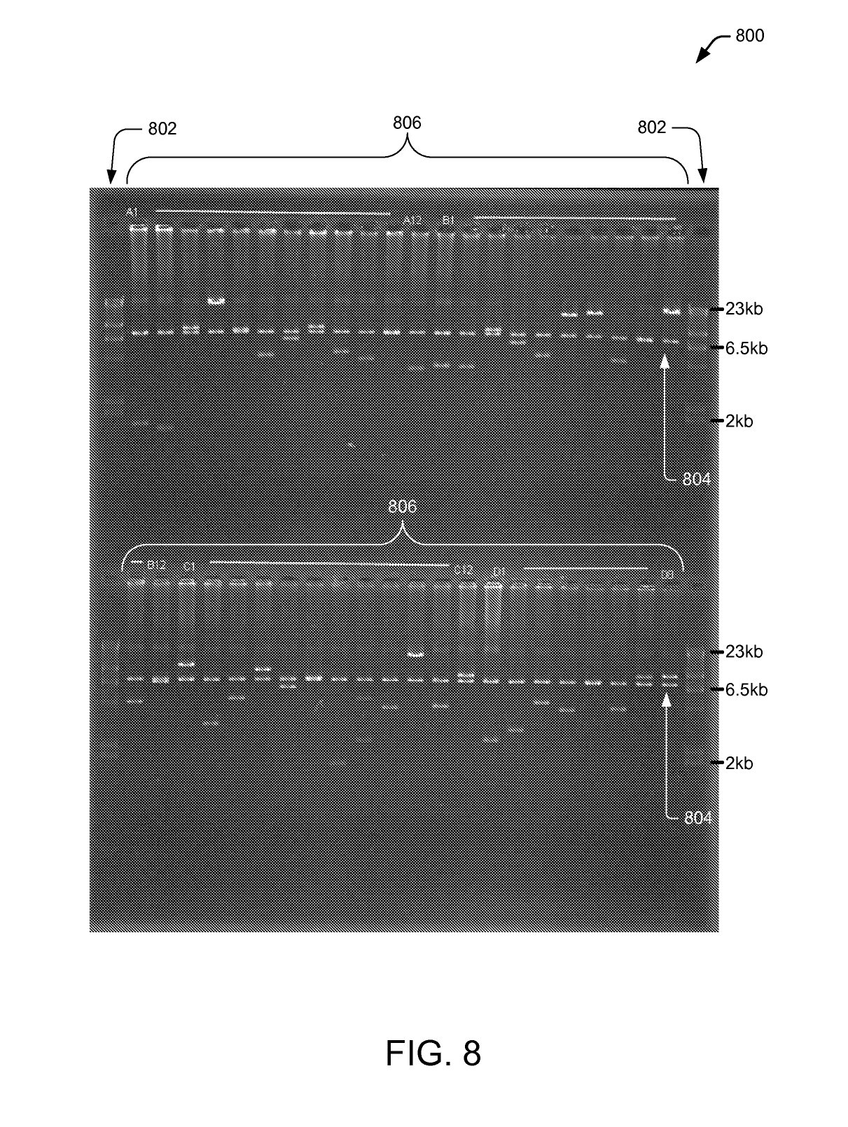

FIG. 8. shows a gel 800 generated by the above procedure. Lanes 802 include ladders generated by HindIII digest of lambda DNA (λH₃) with marker bands from 2 kb to 23 kb. A band 804 found in all lanes at approximately 7 kb represents the pECBAC1 vector (cut at two NotI sites). Other bands observed on the gel are of the human gDNA insert (and very short pieces of vector from the NotI sites). This can be visualized as the schematic 600 shown in FIG. 6. The 44 samples loaded in the central lanes 806 of the gel 800 show the presence of gDNA inserts that are on average around 7 kb which is much less than the average 100 kb shown in the gel 700 of Example 1. This indicates that on average 93 kb section of the inserts were removed by the Nsil digestion. Lanes 806 that show more than two bands (e.g., lane C8) indicate that the gDNA insert included NotI sites. When the insert DNA possess no NotI sites, then the gel shows two bands: a vector band and a single insert band, when the insert DNA possess two NotI sites the gel shows three bands: a vector band and two insert bands, when insert DNA possess three NotI sites the gel shows four bands (vector band and three insert bands) and so on.

### Example 3

This example illustrates large insert mate pair sequence data generated by sequencing vectors prepared according to an embodiment of the invention. High molecular weight human genomic DNA was prepared according to the procedures described in Example 1. The gDNA was partially digested with HindIII and ligated into HindIII prepared pECBAC1 vector as described in Example 1. The HindIII ligated gDNA and pECBAC1 were digested to completion with Nsil and re-ligated together using the procedure of Example 2. Without transforming the re-ligated vectors into E. coli, the re-ligated vectors (which include human gDNA mate pair tags) were used as starting templates in DNA sequencing.

Human gDNA mate pair inserts generated from re-circularized vectors were sequenced using a PacBio next-generation DNA sequencing instrument according to instructions provided by the manufacture. The protocol prepares 10 kb libraries from 1 μg to 5 μg of DNA.

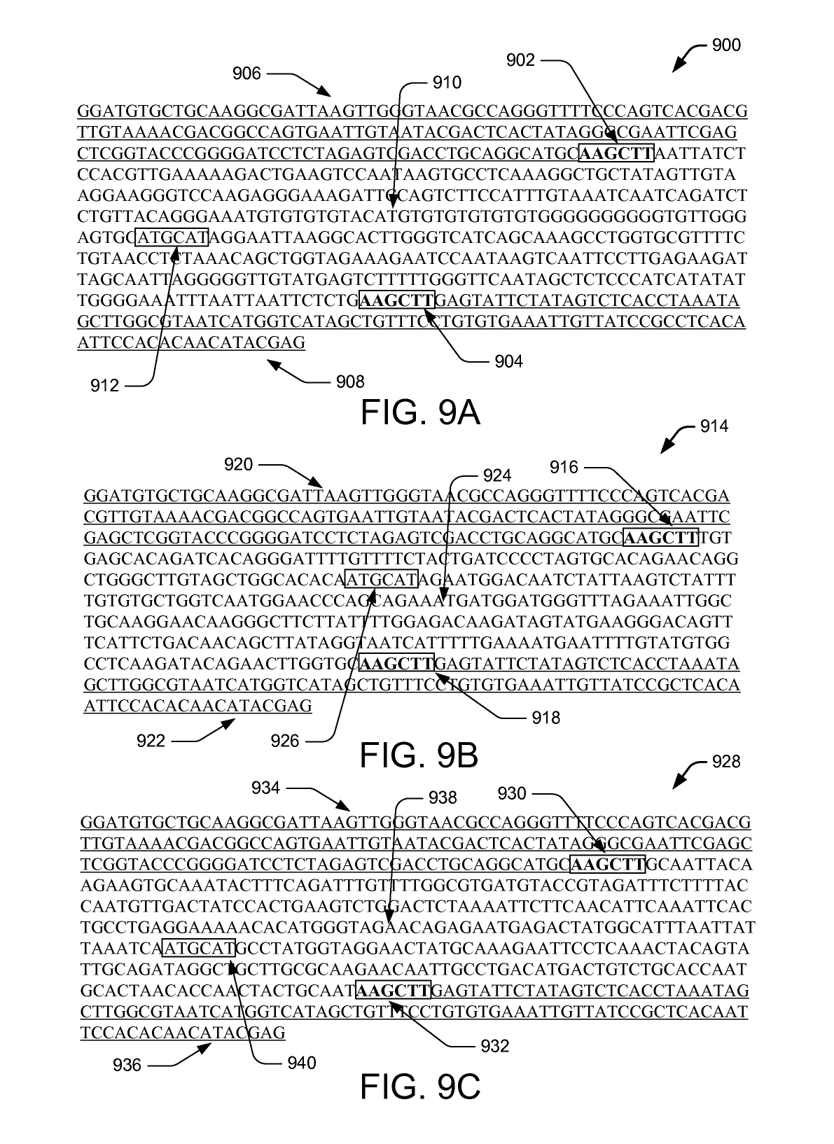

FIG. 9A is a sequence 900 of Homo sapiens chromosome 8, clone RP11-587H10 as identified by Basic Local Alignment Search Tool (BLAST) analysis of sequencing data. The sequence 900 includes two HindIII restriction sites 902 and 904 where the vector DNA 906 and 908 is ligated to the insert DNA 910. A Nsil restriction site 912 is present near the middle of the insert DNA 910. The portion of the insert DNA 910 from the HindIII restriction site 902 to the Nsil restriction site 912 corresponds to a first end of the high-molecular weight gDNA insert. The portion of the insert DNA 910 from the Nsil restriction site 912 to the HindIII restriction site 904 corresponds to a second end of the high-molecular weight gDNA insert. The length of the gDNA originally present between the first and second ends of the high-molecular weight gDNA insert is about 97 kb based on comparison to published sequence data for human chromosome 8.

FIG. 9B is a sequence 914 of Homo sapiens chromosome 3, clone RP11-25D11 as identified by BLAST analysis of sequencing data. The sequence 914 includes two HindIII restriction sites 916 and 918 where the vector DNA 920 and 922 is ligated to the insert DNA 924. A Nsil restriction site 926 is present near the middle of the insert DNA 924. The portion of the insert DNA 924 from the HindIII restriction site 916 to the Nsil restriction site 926 corresponds to a first end of the high-molecular weight gDNA insert. The portion of the insert DNA 924 from the Nsil restriction site 926 to the HindIII restriction site 918 corresponds to a second end of the high-molecular weight gDNA insert. The length of the gDNA originally present between the first and second ends of the high-molecular weight gDNA insert is about 96 kb based on comparison to published sequence data for human chromosome 3.

FIG. 9C is a sequence 928 of Homo sapiens chromosome 1 that covers a region that is not found in any published DNA sequence as identified by BLAST analysis of sequencing data. The sequence 928 includes two HindIII restriction sites 930 and 932 where the vector DNA 934 and 936 is ligated to the insert DNA 938. A Nsil restriction site 940 is present near the middle of the insert DNA 938. The portion of the insert DNA 938 from the HindIII restriction site 930 to the Nsil restriction site 940 corresponds to a first end of the high-molecular weight gDNA insert. The portion of the insert DNA 938 from the Nsil restriction site 940 to the HindIII restriction site 932 corresponds to a second end of the high-molecular weight gDNA insert. The length of the gDNA originally present between the first and second ends of the high-molecular weight gDNA insert is unknown because there identified sequence does not match any published sequence data.

1. A method of preparing a double-stranded polynucleotide for sequencing, the method comprising:
digesting a first circular nucleic acid molecule with a restriction endonuclease, the first circular nucleic acid molecule comprising a vector lacking restriction sites for the restriction endonuclease and an insert having at least two restriction sites for the restriction endonuclease; and
ligating a first end of the insert cut by the restriction endonuclease to a second end of the insert cut by the restriction endonuclease to create a second circular nucleic acid molecule comprising the first end of the insert, the second end of the insert, and the vector.

2. The method of claim 1, wherein the digesting comprises digesting to completion.

3. The method of claim 1, wherein the restriction endonuclease comprises a restriction endonuclease that recognizes a corresponding restriction site on methylated DNA.

4. The method of claim 1, wherein the restriction endonuclease is selected from the group comprising Nsil and EcoRI.

5. The method of claim 1, wherein the vector is a DNA vector capable of holding an insert sequence longer than about 20 kb.

6. The method of claim 1, wherein the vector is selected from the group comprising bacterial artificial chromosomes (BAC), yeast artificial chromosomes (YAC), P1-derived artificial chromosomes (PAC), and transformation-competent artificial chromosomes (TAC).

7. The method of claim 1, wherein the insert comprises at least one of genomic DNA, mitochondrial DNA, or chloroplast DNA.

8. The method of claim 1, wherein the digesting and the ligating occur in a same buffer solution.

9. The method of claim 1, wherein the digesting and the ligating occur without introducing the first circular nucleic acid molecule or the second nucleic acid molecule into a host cell.

10. The method of claim 1, further comprising, following the digesting and prior to the ligating, inactivating or removing the restriction endonuclease.

11. A method of preparing a mate pair, the method comprising:
digesting a cloning vector construct with a restriction endonuclease that recognizes a corresponding restriction site on methylated genomic DNA, the cloning vector construct comprising a cloning vector lacking a restriction site for the restriction endonuclease and a genomic DNA insert having at least two restriction sites for the restriction endonuclease; and
ligating a first end of the genomic DNA insert cut by the restriction endonuclease to a second end of the genomic DNA insert cut by the restriction endonuclease thereby re-ligating the cloning vector construct omitting a middle portion of the genomic DNA.

12. The method of claim 11, wherein the cloning vector is selected from the group comprising bacterial artificial chromosomes (BAC), yeast artificial chromosomes (YAC), P1-derived artificial chromosomes (PAC), and transformation-competent artificial chromosomes (TAC).

13. The method of claim 11, wherein the cloning vector is selected from the group comprising pECBAC1, pBELO11, pCC1BAC, pindigoBAC-5, and pBACe36.

14. The method of claim 11, wherein the restriction endonuclease is selected from the group comprising Nsil and EcoRI.

15. The method of claim 11, wherein the digesting and the ligating occur in a same buffer solution.

16. The method of claim 11, wherein the digesting and the ligating occur without introducing the cloning vector construct into a cell.

17. The method of claim 11, further comprising, following the digesting and prior to the ligating, inactivating or removing the restriction endonuclease.

18. A circular nucleic acid molecule comprising:
a vector capable of stably holding DNA inserts of at least about 20 kb, the vector lacking a restriction site for a restriction endonuclease and lacking an origin of replication;
a first end of a DNA fragment; and
a second end of the DNA fragment ligated to the first end of the DNA fragment at a restriction site of the restriction endonuclease, wherein a middle portion of the DNA fragment between the first end of the DNA fragment and the second end of the DNA fragment is omitted.

19. The circular nucleic acid molecule of claim 18, wherein the DNA fragment comprises at least one of genomic DNA, mitochondrial DNA, or chloroplast DNA.

20. The circular nucleic acid molecule of claim 18, wherein the first end of the DNA fragment, the second end of the DNA fragment, or both are at least about 1 kb.
